(19) United States
(12) Patent Application Publication
(10) Pub. No.: US 2018/0247702 A1
(43) Pub. Date: Aug. 30, 2018

(54) SECURE, ACCURATE, AND EFFICIENT PATIENT INTAKE SYSTEMS AND METHODS

(71) Applicant: Michigan Health Information Network Shared Services, East Lansing, MI (US)

(72) Inventors: Timothy A. PLETCHER, Mount Pleasant, MI (US); Jeffrey A. LIVESAY, Winston-Salem, NC (US)

(73) Assignee: Michigan Health Information Network Shared Services, East Lansing, MI (US)

(21) Appl. No.: 15/961,605

(22) Filed: Apr. 24, 2018

Related U.S. Application Data
(63) Continuation-in-part of application No. 14/643,910, filed on Mar. 10, 2015.
Application No. 14/643,910 is a continuation-in-part of application No. 15/855,319, filed on Dec. 27, 2017.
Application No. 15/855,319 is a continuation-in-part of application No. 15/847,506, filed on Dec. 19, 2017.

(51) Int. Cl. G16H 10/60; G16H 80/00; G06Q 50/26; G16H 10/20

(52) U.S. Cl. CPC G16H 10/60; G16H 10/20; G06Q 50/265; G16H 80/00

(57) ABSTRACT

Techniques for intaking a patient at a provider are disclosed. In one particular embodiment, the techniques may be realized as a method for intaking a patient at a provider according to a set of instructions stored on a memory of a computing device and executed by a processor of the computing device comprising the steps of: presenting, on a display of the computing device, an electronic form including a plurality of fields to be populated with information about the patient; prompting the patient to serially enter a plurality of demographic identifiers; determining a match in a master person index between the patient and an identity of a person stored in the master person index; requesting, from the master person index, a common key identifier pertaining to the patient and information about the patient; and prepopulating one or more of the plurality of fields with information from the master person index.

### CROSS-REFERENCE TO RELATED APPLICATIONS

This patent application is a continuation-in-part of and claims priority to U.S. patent application Ser. No. 14/643,910, filed Mar. 10, 2015, entitled Methods and Systems for Common Key Services, U.S. patent application Ser. No. 15/855,319, filed Dec. 27, 2017, entitled Systems and Methods for Facilitating Communication of Health Information, and U.S. patent application Ser. No. 15/847,506, filed Dec. 19, 2017, entitled Systems and Methods for Predicting Healthcare Outcomes Based on Semantic Relationships, each of which is hereby incorporated by reference herein in its entirety.

This patent application is related to U.S. patent application Ser. No. 14/949,395, filed Nov. 23, 2015, now U.S. Pat. No. 9,491,160, which is a continuation-in-part of U.S. patent application Ser. No. 14/642,092, filed Mar. 9, 2015, now U.S. Pat. No. 9,197,638, and a continuation-in-part of U.S. patent application Ser. No. 14/643,910, filed Mar. 10, 2015. U.S. patent application Ser. No. 14/642,092 and U.S. patent application Ser. No. 14/949,395 are hereby incorporated by reference herein in their entirety.

### FIELD OF THE DISCLOSURE

The present disclosure relates generally to patient information and, more particularly, to secure, accurate, and efficient patient intake systems and methods.

### BACKGROUND OF THE DISCLOSURE

Patient information, demographic and medical, is required and utilized in the health care industry for various purposes. A health organization or a healthcare provider typically requires a new patient to fill out, review, and/or sign one or more intake forms regarding the patient's medical history, prescriptions, HIPAA (Health Insurance Portability and Accountability Act) acknowledgment, release of information, financial policy, right of access, health risk assessment, etc. A new patient may also have to fill out, review, and/or sign various types of consent forms related to mental/behavioral health, research, clinical trial, advance directives (e.g., living will, do not intubate, do not resuscitate, organ donor, etc.), etc.

Today, the vast majority of these intake and consent forms are paper-based, and there is generally significant duplication of patient information across these forms and across health organizations and providers. For instance, a new patient must fill out her name, date of birth, address, etc., repeatedly on multiple forms. Whenever a patient is seen at a new health organization or by a new provider, the patient is very likely to fill out forms that are the same as or similar to those that she has previously filled out at another health organization or provider. A new patient also carries the burden of knowing and remembering current and previous medical conditions, surgeries, diagnoses, treatments, prescriptions, etc., and any corresponding dates. Moreover, patient information written on these forms are typically recorded by humans into patient databases via keyboard-entries. Such processes and systems are clearly inefficient, costly, and prone to mistakes.

In view of the foregoing, it may be understood that there may be significant problems and shortcomings associated with current systems and methods of recording and sharing patient information. Accordingly, there is a need for systems and methods that allow patients to electronically create intake forms once and enter every piece of information once, and that allow secure communication and accurate sharing of the patient's information regardless of geographical location of health organizations or providers.

### SUMMARY OF THE DISCLOSURE

Technique for intaking a patient at a provider are disclosed. In one particular embodiment, the techniques may be realized as a method for intaking a patient at a provider according to a set of instructions stored on a memory of a computing device and executed by a processor of the computing device comprising the steps of: presenting, on a display of the computing device, an electronic form including a plurality of fields to be populated with information about the patient; prompting the patient to serially enter, via one of a graphical user interface and a keyboard, a plurality of demographic identifiers; determining, after the patient enters each of the plurality of demographic identifiers and based on the one or more demographic identifiers entered, a match in a master person index between the patient and an identity of a person stored in the master person index; requesting, from the master person index, a common key identifier pertaining to the patient and information about the patient; and prepopulating one or more of the plurality of fields with information from the master person index.

In accordance with other aspects of this particular embodiment, the requesting step may include invoking a common key service.

In accordance with other aspects of this particular embodiment, the method may further comprise determining, based on the common key identifier, a relationship of the patient with one or more other providers; querying the one or more other providers for additional information about the patient; and prepopulating one or more of the plurality of fields with the additional information.

In accordance with other aspects of this particular embodiment, the method may further comprise prompting the patient to populate empty fields out of the plurality of fields; and prompting the patient to update the prepopulated fields.

In accordance with other aspects of this particular embodiment, the determining step may include invoking an active care relationship service.

In accordance with other aspects of this particular embodiment, the querying step may include: retrieving, from a provider directory utility, one or more electronic addresses for the one or more other providers; and invoking an intelligent query broker to query the one or more other providers for information about the patient.

In accordance with other aspects of this particular embodiment, the method may further comprise prompting the patient to populate empty fields out of the plurality of fields; and prompting the patient update the prepopulated fields.

In accordance with other aspects of this particular embodiment, the method may further comprise invoking an identity proofing service to verify the identity of the patient.

In accordance with other aspects of this particular embodiment, the electronic form is one of an intake form and a consent form, wherein the intake form relates to one of the patient's medical history, prescriptions, HIPAA (Health Insurance Portability and Accountability Act) acknowledgment, release of information, financial policy, right of access, and health risk assessment; and the consent form relates to one of mental/behavioral health, research, clinical trial, and advance directives.

In accordance with other aspects of this particular embodiment, wherein the computing device is one of a desktop computer, a laptop computer, a tablet, a smartphone, and a server.

In another particular embodiment, the techniques may be realized as a non-transitory computer readable medium having instructions stored thereon that, upon execution by a processor of a computing device, causes the computing device to perform operations, wherein the instructions comprise the above-recited steps.

The present disclosure will now be described in more detail with reference to particular embodiments thereof as shown in the accompanying drawings. While the present disclosure is described below with reference to particular embodiments, it should be understood that the present disclosure is not limited thereto. Those of ordinary skill in the art having access to the teachings herein will recognize additional implementations, modifications, and embodiments, as well as other fields of use, which are within the scope of the present disclosure as described herein, and with respect to which the present disclosure may be of significant utility.

### BRIEF DESCRIPTION OF THE DRAWINGS

In order to facilitate a fuller understanding of the present disclosure, reference is now made to the accompanying drawings, in which like elements are referenced with like numerals. These drawings should not be construed as limiting the present disclosure, but are intended to be illustrative only.

FIG. 1 illustrates an exemplary system for collecting, maintaining, and updating patient information in accordance with an embodiment of the present disclosure.

FIG. 2 is a block diagram illustrating an exemplary computing device in accordance with an embodiment of the present disclosure.

FIG. 3 is a flow diagram illustrating a method for generating a common key for known persons in accordance with an embodiment of the present disclosure.

FIG. 4 is a flow diagram illustrating a method for generating a common key for unknown persons in accordance with an embodiment of the present disclosure.

FIG. 5 is a flow diagram illustrating a method for utilizing a known common key for a known person in accordance with an embodiment of the present disclosure.

FIG. 6 is a flow diagram illustrating a method for acquiring records using a common key in accordance with an embodiment of the present disclosure.

FIG. 7 is a flow diagram illustrating a method for verifying potential person matches in accordance with an embodiment of the present disclosure.

FIG. 8 is a flow diagram illustrating a method for updating files using a common key service in accordance with an embodiment of the present disclosure.

FIG. 9 is a flow diagram illustrating a method for utilizing a common key service in conjunction with a patient's hospital visit in accordance with an embodiment of the present disclosure.

FIG. 10 is a flow diagram illustrating a method for utilizing a common key service in accordance with an embodiment of the present disclosure.

FIG. 11 is a flow diagram illustrating a new patient's intake process in accordance with an embodiment of the present disclosure.

### DETAILED DESCRIPTION OF EMBODIMENTS

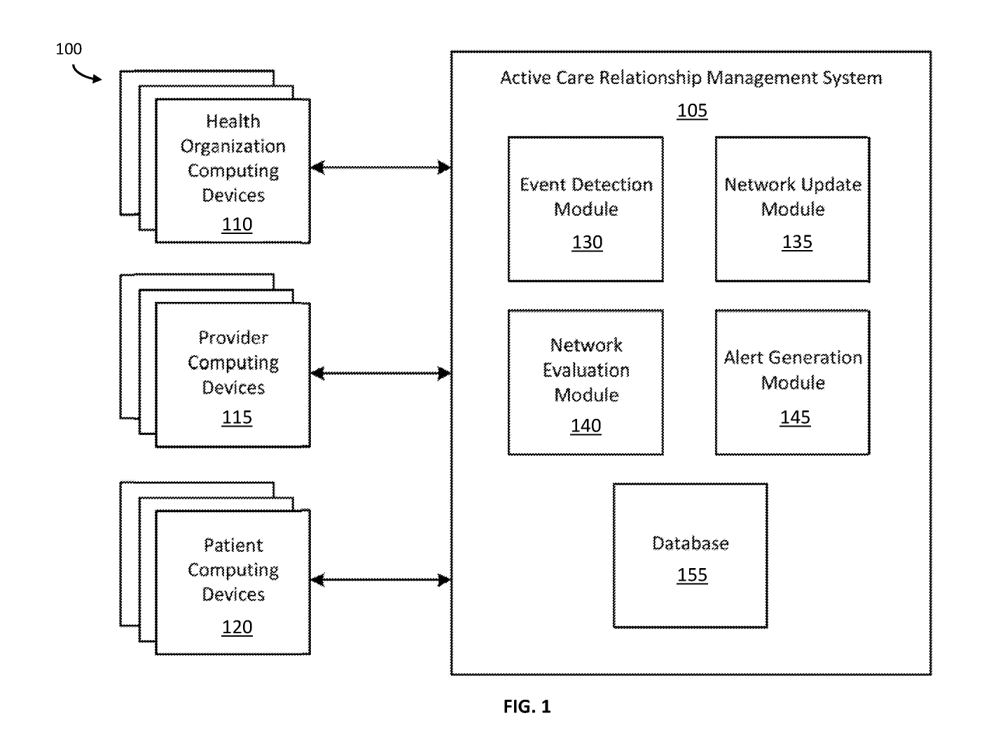

Referring to FIG. 1, there is shown an exemplary system 100 for collecting, maintaining, and updating patient information in accordance with an embodiment of the present disclosure.

The system 100 includes an active care relationship management (ACRM) system 105 that may be used to provide an active care relationship service (ACRS), a plurality of health organization computing devices 110, a plurality of provider computing devices 115, and a plurality of patient computing devices 120. The ACRM system 105 includes an event detection module 130, a network update module 135, a network evaluation module 140, an alert generation module 145, and a database 155.

The ACRM system 105 is configured to assist with the collection, maintenance, and distribution of patient information. For example, the ACRM system 105 may receive patient information from the health organization computing devices 110, the provider computing devices 115, and the patient computing devices 120, and may process the received information to determine relationships between various entities, including patients, physicians, and other healthcare providers. The ACRM system 105 may generate and continuously update a semantic network based on the information received from the health organization computing devices 110, the provider computing devices 115, and the patient computing devices 120. The semantic network may be an interconnection of nodes, which may include patients, medications, medical conditions, providers, hospitals, clinics, etc., as described in U.S. patent application Ser. No. 15/847,506, which is incorporated herein in its entirety.

In some embodiments, the generation and update of the semantic network may be carried out by the event detection module 130 and the network update module 135. For example, the event detection module 130 may parse information received from the health organization computing devices 110, the provider computing devices 115, and the patient computing devices 120, and determine whether such information constitutes an event necessitating an update to the semantic network. The network update module 135 may add a node corresponding to either a provider or a patient to the semantic network if such nodes are not already present in the semantic network, and may add or modify the interconnection among the nodes. The network update module 135 also may store semantic information for the node interconnection. The network evaluation module 140 may analyze the semantic network to make inferences that may be relevant to the healthcare of one or more patients in the network. For example, the network evaluation module 140 may be configured to recognize patterns that may be used to predict healthcare outcomes or to select preventive care strategies for patients. The alert generation module 145 may be configured to provide alerts, such as an email, a text message, a phone call, or a mobile application notification, to any of the health organization computing devices 110, the provider computing devices 115, or the patient computing devices 120. The database 155 may store information about the semantic network, consent rules, policies, etc.

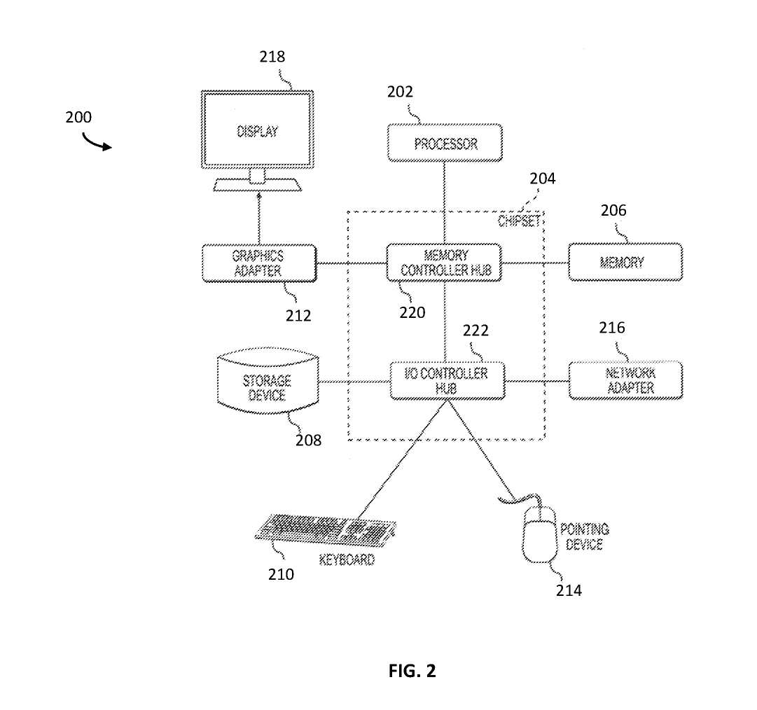

In some embodiments, the health organization computing devices 110 may be any type or form of computing device owned, operated, or otherwise accessed by a health organization, such as a health system, a hospital, a health plan, a medical insurer, a prescription benefit manager, a pharmacy, or a clinic. In some arrangements, each of the health organization computing devices 110 is implemented as at least one of a server, a desktop computer, or a laptop computer. An exemplary computing device will be described below with respect to FIG. 2. In some other arrangements, each of the health organization computing devices 110 may be a mobile computing device such as a tablet computing device, or a handheld computing device, such as a smartphone that may be accessed by an employee or other representative of the respective health organization.

Similarly, the provider computing devices 115, the patient computing devices 120, and the ACRM system 105 may also be any type or form of computing device owned, operated, or otherwise accessed by a provider, a patient, and a ACRS provider, respectively. In general, a provider may include a physician, a pharmacist, or any other person or group of people that provides healthcare to patients. In some embodiments, the provider computing devices 115, the patient computing devices 120, or the ACRM system 105 may be implemented as at least one of a server, a desktop computer, a laptop computer, a tablet computing device, or a handheld computing device.

FIG. 2 is a block diagram illustrating an exemplary computing device 200 in accordance with an embodiment of the present disclosure. In alternative embodiments, fewer, additional, and/or different components may be present. The computing device 200 may be any computing machine, such as a tablet, smart phone, laptop computer, desktop computer, server, remote client device, gaming device, smart television device, wearable computer, or any combination thereof. The computing device 200 may include at least one processor 202 coupled to a chipset 204. The chipset 204 may include a memory controller hub 220 and an input/output (I/O) controller hub 222. A memory 206 and a graphics adapter 212 may be coupled to the memory controller hub 220, and a display 218 may be coupled to the graphics adapter 212. A storage device 208, a keyboard 210, a pointing device 214, and a network adapter 216 may be coupled to the I/O controller hub 222. Other embodiments of the computing device 200 may have different architectures.

The storage device 208 may be a non-transitory computer-readable storage medium such as a hard drive, compact disk read-only memory (CD-ROM), DVD, or a solid-state memory device (e.g., read only memory (ROM) and random access memory (RAM)). The memory 206 may hold instructions and data that may be used by the processor 202. The pointing device 214 may be a mouse, a track ball, or other type of pointing device, and may be used in combination with the keyboard 210 to input data into the computing device 200. The pointing device 214 may also be a gaming system controller, or any type of device used to control a gaming system. For example, the pointing device 214 may be connected to a video or image capturing device that employs biometric scanning to detect a specific user. The specific user may employ motion or gestures to command the point device 214 to control various aspects of the computing device 200. The graphics adapter 212 may display images and other information on the display 218. To enhance interaction with a user, the herein disclosed embodiments may be implemented using an interactive display, such as a graphical user interface (GUI). Such GUIs may include interactive features such as pop-up or pull-down menus or lists, selection tabs, and other features that may receive human inputs. The network adapter 216 may couple the computer system device 200 to one or more computer networks.

The computing device 200 may be adapted to execute one or more computer programs for providing functionality described herein. A computer program (also known as a program, module, engine, software, software application, script, or code) may be written in any form of programming language, including compiled or interpreted languages, declarative or procedural languages, and the program may be deployed in any form, including as a stand-alone program or as a module, component, subroutine, object, or other unit suitable for use in a computing environment. A computer program may, but need not, correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program may be deployed to be executed on one computing device 200 or on multiple computing devices 200 that may be located at one site or distributed across multiple sites and interconnected by a communication network. In one embodiment, program modules may be stored into the storage device 208, loaded into the memory 206, and executed by the processor 202.

As used herein, the term module refers to computer program logic used to provide the specified functionality. Thus, a module may be implemented in hardware, firmware, and/or software. As used herein, the term processor encompasses all kinds of apparatus, devices, and machines for processing data, including by way of example a programmable processor, a computer, a system on a chip, or multiple ones, or combinations, of the foregoing. The processor may include special purpose logic circuitry, e.g., an FPGA (field programmable gate array) or an ASIC (application-specific integrated circuit). The processor also may include, in addition to hardware, code that creates an execution environment for the computer program in question, e.g., code that constitutes processor firmware, a protocol stack, a database management system, an operating system, a cross-platform runtime environment, a virtual machine, or a combination of one or more of them.

The types of computing devices used by the entities and processes disclosed herein may vary depending upon the embodiment and the processing power required by the entity. The computing device 200 may be a mobile device, tablet, smartphone or any sort of computing element with the above-listed elements. For example, a data storage device, such as a hard disk, solid state memory or storage device, may be stored in a distributed database system comprising multiple blade servers working together to provide the functionality described herein. The computer devices may lack some of the components described above, such as keyboards 210, graphics adapters 212, and displays 218.

Multiple computing devices 200 may be clustered to form a computing system of clients and servers. A client and server are generally remote from each other and typically interact through a communications network. The relationship of client and server arises by virtue of computer programs running on the respective computing devices and having a client-server relationship to each other. In some embodiments, a server transmits data (e.g., an HTML page) to a client (e.g., for purposes of displaying data to and receiving user input from a user interacting with the client device). Data generated at the client (e.g., a result of the user interaction) may be received from the client at the server.

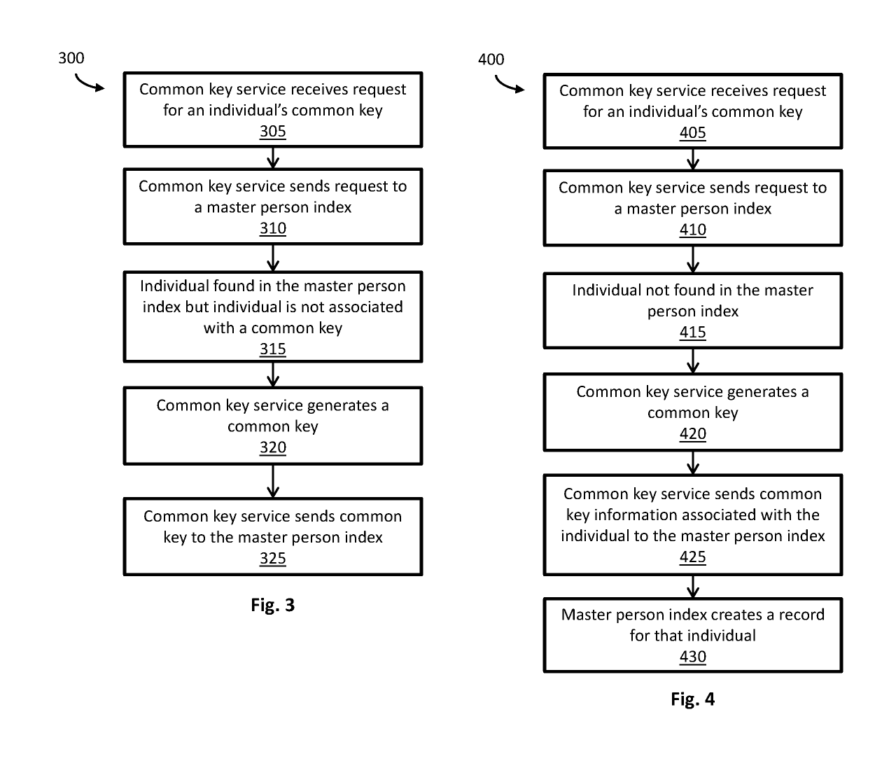

The methods of generating and utilizing common keys in a common key service, described below with respect to FIGS. 3-11, may be performed partially or wholly on a processor, such as the one described above with regards to the computing device 200. A common key provides a way to match patients and their records across multiple organizations, applications, and services, such as an ACRS supported by an ACRM system (e.g., the ACRM system 105). With a common key, full and complete records for a patient may be accessed or modified as desired. Even if multiple records are associated with a patient, a common key may link the various records together. A common key service is safe and secure, protects confidentiality, and has high accuracy and data integrity of records shared across the multiple entities.

In an embodiment of the present disclosure, exchange of information between health organizations and providers may be performed utilizing common key services. Each patient may be matched to a single identity, and that identity is assigned a unique common key. The common key may be an alphanumeric sequence. The common key is unique to each patient, and is used to link records stored, housed, and/or generated by multiple health organizations and providers.

The common key, when associated with the single identity of a patient, may be used to associate records of the patient across multiple entities and data stores across the healthcare market, such as primary care physician records, specialist records, hospital records, demographic records, billing information records, insurance information records, medical history records, care coordinator records, research databases, oncological databases, behavioral health system databases, pharmacy databases, etc.

A common key service may be used to link patients to their respective electronic medical records. Records may come from various formats and be stored across disparate systems. A common key may be used to link various records of a patient together. Additionally, a patient's demographic information (e.g., address, name, billing information, etc.) may change over time, making demographic information outdated or subject to error. By using a common key to keep track of records, complications and errors in treating patients due to incomplete information and records may be reduced.

A common key service may minimize mismatched record/patient associations and ensure that the record keeping is accurate. Matches and links between a patient and the patient's records may be affected across multiple organizations, applications, and services. This may lead to higher data integrity, which in turn improves patient safety by minimizing mistakes made by those utilizing the data. For example, a common key service may provide enhanced patient safety and care coordination by ensuring that medication and allergy information is tied to the correct person through a continuum of care, enhancing patient safety and potentially reducing the risk of medical errors. A common key service may also reduce work completed to coordinate care to patients across different organizations, applications, and services. This may also reduce cost of record keeping in service industries, such as health care. Furthermore, a common key service may be implemented to utilize current health information exchanges (HIE) and healthcare information technology (HIT) systems.

In an embodiment of the present disclosure, an HIT or HIE endpoint may be mapped to a master person index (MPI) using common key services. For example, a governmental body such as a state government may maintain an MPI. The MPI may contain a record of every person in the state or known to the state. Such an MPI may be populated using information acquired through different government entities, such as entities that issue identification, process taxes, process health benefits, or schools. The common key service may insure that any medical records are mapped to a single identity of an individual as maintained on the MPI. In one embodiment of the present disclosure, the systems and methods disclosed herein for a common key service may be executed as a web service that utilizes application programming interface (API) calls to the MPI and/or HITs and HIEs. Such an embodiment may allow for easy integration of an MPI, HIT, and/or HIE with the common key service.

In one embodiment of the present disclosure, the common key service may be used in a case that tracks an active care relationship of a provider or providers with a patient in the healthcare industry. For example, a Patient X is admitted to a hospital. The hospital may be a part of a data sharing organization (DSO). Such a DSO may include other health care providers or hospitals that are a part of a single health system. A provider, such as a physician, at the hospital may generate an admit-discharge-transfer (ADT) notification that indicates Patient X has been admitted to the hospital. The ADT notification may be sent to an ACRS supported by an ACRM system (e.g., ACRM system 105) via the DSO that invokes the common key service. In other words, when the DSO attempts to store the ADT notification, the ACRS (which may be offered separately from or in conjunction with the common key service) will invoke or reference a common key from the common key service to determine and/or verify that the ADT notification is properly associated with Patient X and stored properly.

The common key service then utilizes demographic information received in conjunction with the ADT notification to search an MPI for the identity of Patient X. The MPI may be maintained by a state agency, as indicated above, or may be stored and maintained by another entity, such as the entity offering the common key service. If a match for Patient X is found in the MPI and Patient X is already associated with a common key, the MPI sends back to the common key service Patient X′s unique common key. In this way, the ADT notification may be properly recorded and associated with the true identity of Patient X in the active care relationship files. Similarly, the common key may also be used by the DSO and the ACRS to determine other records relating to Patient X, which may facilitate proper care to Patient X while Patient X is treated at the hospital.

If the MPI finds a true identity of Patient X based on the demographic information, but Patient X is not yet associated with a unique common key, the MPI will request a common key from the common key service. A common key is generated by the common key service and that common key is assigned to Patient X. Similar to the above, the common key may then be added to the appropriate records. If the MPI does not find an identity match for Patient X or a common key, the MPI may create a new person record and request a new unique common key from the common key service. Examples of persons that may not have a record or common key may be a newborn or a person that has no previous affiliation with the body maintaining the MPI.

In another example, Patient X may be admitted to a hospital and the DSO associated with the hospital may already be aware of a common key associated with Patient X. Accordingly, when an ADT notification is generated, the DSO may send the ADT notification to the ACRS as well as the common key service along with the known common key. Accordingly, the records may be stored and associated with the common key, and the common key service may not have to look up the common key and match an identity in the MPI. However, in an alternative embodiment, the common key service may still look up the common key and the identity of Patient X in the MPI so as to verify that all of the information from the DSO is correct.

In another embodiment of the present disclosure, the common key service may be utilized to anonymize and make records more secure and private. For example, after a common key has been established for a patient and known by DSOs, health care providers, an MPI, the common key service, etc., personal identifying information about the patient may no longer be transmitted between those various entities. For example, a patient's records may be updated using only the patient's common key to identify which records to update, and the patient's name, birthdate, social security number, address, other demographic data, etc. may not be used to update the patient's medical records. In this way, the transmission of the patient's medical records and the medical records themselves may be de-identified.

In another embodiment of the present disclosure, a different use case may be applied in conjunction with a common key service. In the embodiment, a researcher may be studying a medical condition and utilizing records to perform a study. For example, the researcher may be studying the effectiveness of depression treatment as a cancer patient progresses through chemotherapy. Different treatments for a cancer patient may be administered by multiple providers for a single patient. Records for the different treatment may be generated and/or stored on multiple different systems. Accordingly, locating accurate information across disparate systems for a single patient may be difficult. For example, the name John Smith is very common, and if he is a subject of the study, linking together different medical records with various medical records numbers may be difficult. In other words, the researcher may find it difficult to piece together exactly which John Smith got what treatments, when the treatments were administered, and where the treatments were administered because, in part, each system may utilize different medical record numbers for the various John Smiths that exist. Accordingly, the researcher may utilize the common key system to determine which of the various records relating to a John Smith related to the John Smith that is the subject of the study. In this way, the research may be more easily and accurately conducted. In one embodiment, the records may not be associated with a common key, so the common key service matches each record to a true identity associated with the John Smith that is the subject to the study. In another embodiment, the researcher may find records that are already associated with a common key. In this instance, the researcher may utilize the common key service to request the common key of the John Smith that is the subject of the study. Once the common key for the correct John Smith is determined, the records that include the same common key may be easily identified, either by the researcher or automatically by the common key service. In other words, this embodiment allows researchers to link information from various systems to the appropriate patients using the common key services.

The common key services systems and methods disclosed herein overcomes difficulties in matching patients to facilitate the exchange of health information, despite medical information being stored in disparate systems. For example, one hospital registration/admission system may record gender as Male, Female, Unknown, while another hospital system may list M, F, or U instead. Furthermore, inconsistencies in patient demographics may also complicate accurate matching. For example, a patient's name may be entered as Jane Smith-Jones in one system; Jane Smith Jones (without the hyphenation (-)) in another system; and a third system may record her name as Jane Jones. In one system, Jane's address may be her most recent, while another system still has her address as her previous home; one may have her date of birth with year 1975 while the others have her birth year as 1957. In an embodiment of the present disclosure, when a provider or DSO requests records for a particular patient (or records associated with a particular common key), the system may format records according to the way the requesting party stores and transmits patient data and records. In this way, a requesting party may more easily interpret, display, and store the requested information according to the requesting party's system.

To streamline the exchange of information to support meaningful use and accountable care, electronic health care systems utilize reliable matching using a common key service as disclosed herein to determine that the right information is attributed to the right patient every time. The common key services disclosed herein provide a consistent and reliable way to match patients across multiple organizations, applications and services. Such reliable matching capabilities ensure patient safety and high data integrity when data is shared. The common key service links information for individuals or organizations by using best practices for matching criteria to ensure that identifiers and attributes positively and accurately identify multiple types of entities. Examples of attributes that may be used to match identities include demographic information such as name, date of birth, gender, etc. In an alternative embodiment, information unique to the patient such as biometric information (fingerprint, palm scan, eye scan, etc.) may be used to determine an identity match. Individuals and entities that may use a common key service may include patients, beneficiaries, physicians and physician organizations, payers and health plans, hospitals and health systems, health care facilities, public health entities, etc. The common key services disclosed herein may allow accurate data sharing through a wide variety of use cases, such as results delivery, hospital notifications, public health reporting, care coordination and patient safety, quality and administrative reporting, patient engagement, infrastructure (e.g. ACRS, statewide health provider directory, information security/identity management), standard consent, quality assurance systems, etc.

The common key service disclosed herein provides means to link information for individuals or organizations accurately in support of various use cases. For example, improved patient matching increases the volume of outbound ADT messages that accurately reach providers and payers, which helps a widespread health system operate more efficiently. The common key systems and methods disclosed herein may also leverage a state's MPI which may utilize robust processes for managing information about persons and de-duplicating entries with great accuracy.

In an embodiment of the present disclosure, the common key service receives a request for a patient's common key and passes the request to a respective state's MPI. Such a request may result in the following actions: (1) If the patient is found in the MPI, then the common key service creates a common key for the patient and cross-references it with the state's MPI to ensure accurate mapping across systems. (2) If a person is not found in the state's MPI, then the common key service assigns a common key and passes it to the state's MPI, which creates or modifies a record for that patient in the MPI. (3) If a potential match or possible duplicate is identified in the state's MPI the requestor receives a list of possible matches and is prompted to review the records in detail to identify the correct patient and/or to identify errors that caused the duplication in the MPI (e.g., misspelled name, incorrect birth date). The requestor then sends a message to the common key service which informs the MPI as to which of the duplicates is the right person. If the MPI is the source for the duplicate data, an MPI staff may review the data and correct duplicates and errors. Such a process may help ensure that person records are kept up-to-date, improving the integrity of the common key service and making both the MPI and common key service more robust. A record may be modified or created in the MPI by sending a message from the common key service that includes an action and any associated common keys. For example, the action in a message may be one or more of merge, update, or delete. Common keys to merge, update, or delete would be include in the message and associated with the appropriate action in the message.

Additionally, the common key service may utilize an ACRS supported by an ACRM system (e.g., ACRM system 105). An ACRM system or similar records management system may be exposed to the common key service via an API and then mapped to and integrated with the MPI using its APIs. This may yield an ease of technical implementation. For example, a Medicaid population that is serviced using an ACRS may be easily applied and used with a common key service.

FIG. 3 is a flow diagram illustrating a method 300 for generating a common key for known persons in accordance with an embodiment of the present disclosure. In alternative embodiments, fewer, additional, and/or different operations may be performed. Also, the use of a flow diagram is not meant to be limiting with respect to the order of operations performed. In an operation 305, a common key service receives a request for an individual's common key. In an operation 310, the common key service sends a request to an MPI. In an alternative embodiment, the MPI may instead be any database that stores information on persons and de-duplicates records on individual persons. In an operation 315, the individual is found in the MPI, but the individual is not associated with a common key. In an operation 320, the common key service generates a common key. In an operation 325, the common key service sends the common key to the MPI, such that the common key may be associated with the record of the individual in the MPI. In an alternative embodiment, the MPI and the common key service may not be separate systems. In other words, the common key system may store persons similar to an MPI, may determine the person matches in the MPI according to requests from data sharing organization devices or providers, and may use the common keys to de-duplicate records regarding single individuals. In other words, the common key system and the MPI may be the same or multiple systems that achieve the same functions as disclosed herein.

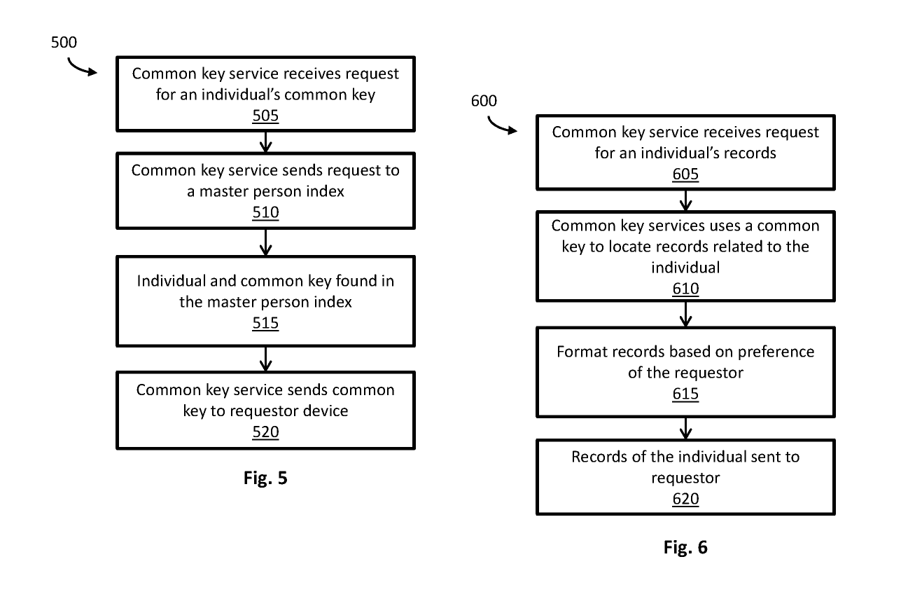

FIG. 4 is a flow diagram illustrating a method 400 for generating a common key for unknown persons in accordance with an embodiment of the present disclosure. In alternative embodiments, fewer, additional, and/or different operations may be performed. Also, the use of a flow diagram is not meant to be limiting with respect to the order of operations performed. In an operation 405, a common key service receives a request for an individual's common key. In an operation 410, the common key service sends a request to an MPI. In an operation 415, the individual is not found in the MPI. In an operation 420, the common key service generates a common key for the individual in response to a record of the individual not being found in the MPI. In an operation 425, the common key service sends the generated common key information associated with the individual to the MPI. In an operation 430, the MPI creates a record for that individual that includes identifying demographic information and the common key. FIG. 5 is a flow diagram illustrating a method 500 for utilizing a known common key for a known person in accordance with an embodiment of the present disclosure. In alternative embodiments, fewer, additional, and/or different operations may be performed. Also, the use of a flow diagram is not meant to be limiting with respect to the order of operations performed. In an operation 505, a common key service receives a request for an individual's common key. In an operation 510, the common key service sends a request to an MPI. In an operation 515, the individual and an associated common key are found in the MPI. In an operation 520, the common key service sends the common key received from the MPI to the requestor device. Additionally, in another embodiment, the common key service may also associate a record from the requestor with the common key.

FIG. 6 is a flow diagram illustrating a method 600 for acquiring records using a common key in accordance with an embodiment of the present disclosure. In alternative embodiments, fewer, additional, and/or different operations may be performed. Also, the use of a flow diagram is not meant to be limiting with respect to the order of operations performed. In an operation 605, the common key service receives a request for an individual's records. In an operation 610, the common key service uses a common key to locate records related to the individual. In one embodiment, the records located may be stored in the common key system. In another embodiment, the records may be located on other systems, such as a DSO system, an ACRM system, a provider system, an MPI, or other locations where records may be stored. The common key utilized to locate or identify the records may be determined in various ways such as the methods described above with respect to FIGS. 3-5. In an operation 615, the records located are formatted based on a format preference of the requestor. This formatting may affect the way certain information/data is presented, and may affect what information/data is presented. In other words, the system may format the delivered records form and determine which records or portions of records should actually be delivered. In an operation 620, the records located and formatted are sent to the requestor.

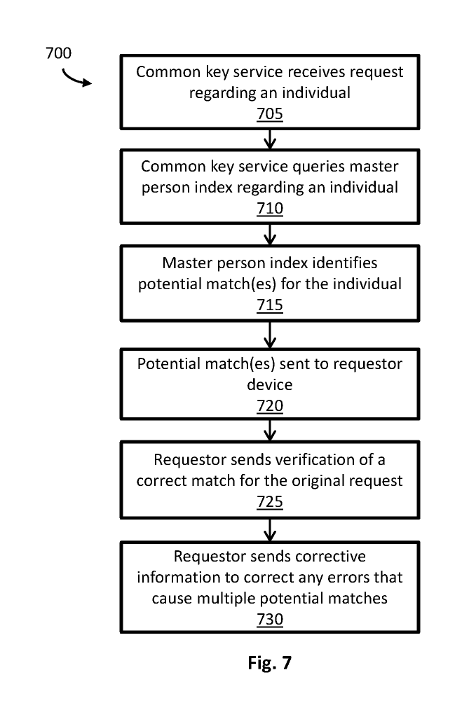

FIG. 7 is a flow diagram illustrating a method 700 for verifying potential person matches in accordance with an embodiment of the present disclosure. In alternative embodiments, fewer, additional, and/or different operations may be performed. Also, the use of a flow diagram is not meant to be limiting with respect to the order of operations performed. In an operation 705, the common key service receives a request regarding an individual. In an operation 710, the common key service queries an MPI regarding the individual. In an operation 715, the MPI identifies potential matches (or a single potential match) for the individual. In an operation 720, the potential match(es) are sent to the requestor device. In an operation 725, the requestor sends verification of a correct match for the original request to the common key service and/or the MPI. In other words, the requestor confirms which of the potential match(es) is the correct match. In an operation 730, the requestor may also send corrective information to correct any errors that cause multiple matches. For example, some demographic information of an individual may be stored incorrectly in the MPI such that the MPI erroneously returned that individual as a potential match. The request may, in the operation 730, correct the error to prevent future mistakes and make the MPI and the common key service more accurate and helpful. In an alternative embodiment, confirmation of a correct match and/or corrective information regarding a record may be sent from an entity or device other than the requestor. For example, the common key service or the MPI may also be able to determine a correct match from potential match(es) and correct incorrect information in a record.

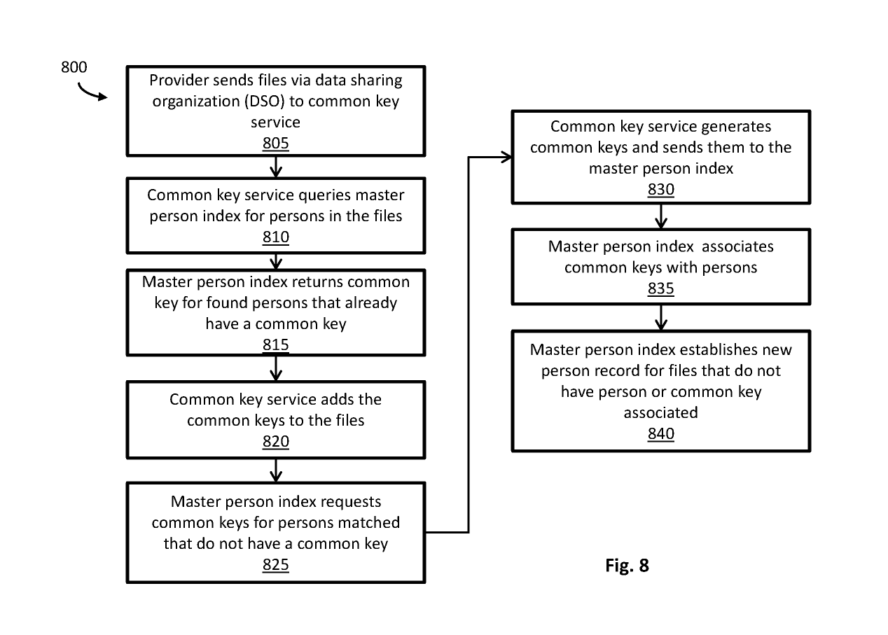

FIG. 8 is a flow diagram illustrating a method 800 for updating files using a common key service in accordance with an embodiment of the present disclosure. In alternative embodiments, fewer, additional, and/or different operations may be performed. Also, the use of a flow diagram is not meant to be limiting with respect to the order of operations performed. In an operation 805, a healthcare provider sends files via a DSO device (e.g., the health organization computing devices 110 or the provider computing devices 115) to a common key service. In an operation 810, the common key service queries an MPI for persons that match persons represented in the files sent from the DSO. In an operation 815, the MPI returns a common key for any matched individuals in the MPI database that match the persons in the files sent from the DSO. In the operation 815, the persons matched have already been assigned a common key. In an operation 820, the common key service adds the common keys attributed to the matched persons to the files. In an operation 825, the MPI requests common keys for persons matched that do not already have a common key assigned. In an operation 830, the common key service generates common keys for those persons and sends the common keys to the MPI. In an operation 835, the MPI associates the generated common keys with the person found in the MPI but previously not yet assigned a common key. In an operation 840, the MPI establishes a new person record for each of the persons from the files that do not yet have a matched person in the MPI or an associated common key. The method demonstrated in FIG. 8 may be utilized to intake and process large amounts of files and records that apply to many varying persons.

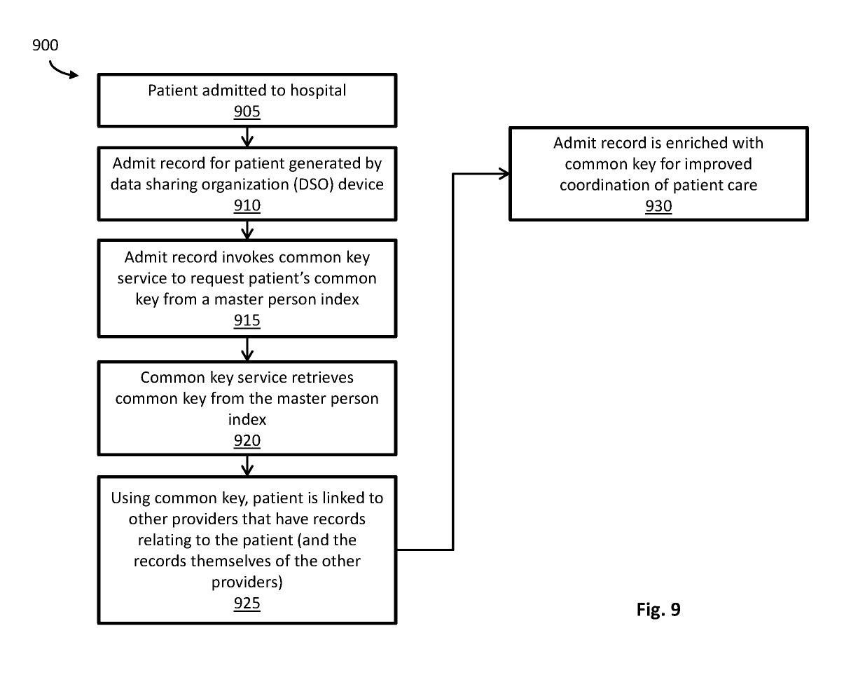

FIG. 9 is a flow diagram illustrating a method 900 for utilizing a common key service in conjunction with a patient's hospital visit in accordance with an embodiment of the present disclosure. In alternative embodiments, fewer, additional, and/or different operations may be performed. Also, the use of a flow diagram is not meant to be limiting with respect to the order of operations performed. In an operation 905, a patient is admitted to a hospital. In an operation 910, an admit record for the patient is generated by a DSO device (e.g., a health organization computing device 110 or a provider computing device 115). In an operation 915, the admit record and the DSO device invokes a common key service to request the patient's common key from an MPI. In an operation 920, the common key service retrieves the patient's common key from the MPI. In alternative embodiments, the patient's common key may be generated similar to the common keys discussed above with respect to FIGS. 3 and 4. In an operation 925, the common key service links the common key to link the patient to other providers that have records relating to that patient (and to the patient's records possessed by the other providers). In an operation 930, the admit record is enriched with the common key for improved coordination of patient care. In another embodiment, the admit record may also be enriched with other information, such as the linked other providers and other provider records identified in the operation 925.

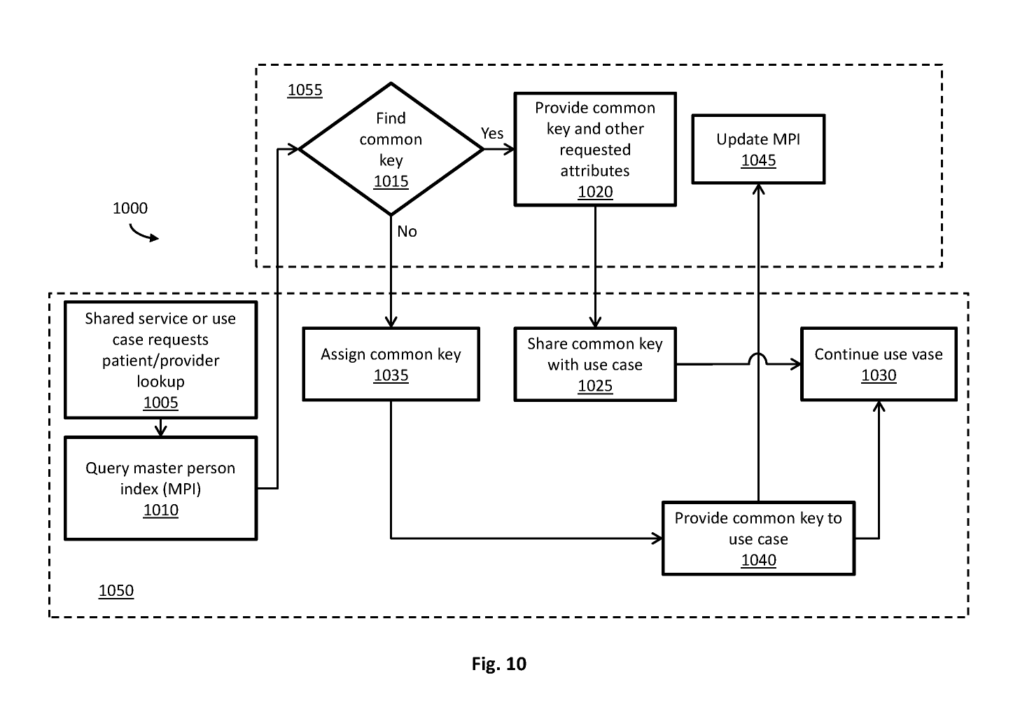

FIG. 10 is a flow diagram illustrating a method 1000 for utilizing a common key service in accordance with an embodiment of the present disclosure. In alternative embodiments, fewer, additional, and/or different operations may be performed. Also, the use of a flow diagram is not meant to be limiting with respect to the order of operations performed. The method 1000 shows steps that may be performed by an MPI 1055, and steps that may be performed by a common key service 1050. In an operation 1005, a shared service or use case requests a patient and/or provider lookup from the common key service 1050. In an operation 1010, the common key service 1050 queries the MPI 1055. In an operation 1015, the MPI 1055 determines whether the patient and/or provider has a common key. If the patient and/or provider does have a common key, the MPI 1055 provides the common key and any other requested attributes (such as demographic information or other records) to the common key service 1050. In an operation 1025, the common key service 1050 in turn provides that information and the common key to the requestor, shared service, use case, etc. In an operation 1030, the common key service 1050 then continues the use case, shared service, etc. In other words, the shared service, use case, etc. utilizes the provided information and/or common key for a purpose for which the information and/or common key was requested, such as obtaining or updating a record. In an operation 1035, the common key service 1050 assigns a common key because the MPI 1055 did not find a common key in the operation 1015. In an operation 1040, the generated common key is provided to the use case, shared service, requestor, etc. In an operation 1045, the generated common key is provided to the MPI 1055, so that the MPI 1055 may be updated with the generated common key. In the operation 1045, the generated common key is associated in the MPI 1055 with the patient and/or provider that the common key has been generated for, such that the common key may be subsequently associated with that patient and/or provider. In the operation 1030, the common key service 1050 then continues the use case, shared service, etc. In other words, the shared service, use case, etc. utilizes the provided information and/or common key for a purpose for which the information and/or common key was requested, such as obtaining or updating a record.

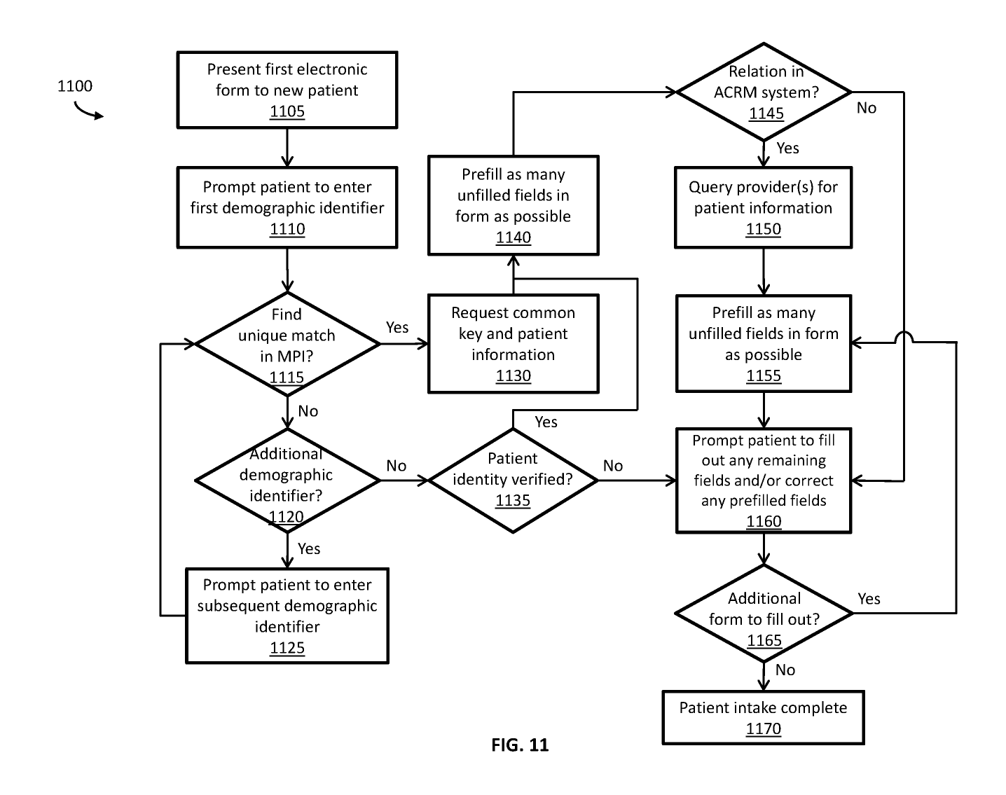

FIG. 11 is a flow diagram illustrating a new patient's intake process 1100 at a health organization or a provider in accordance with an embodiment of the present disclosure. In alternative embodiments, fewer, additional, and/or different operations may be performed. Also, the use of a flow diagram is not meant to be limiting with respect to the order of operations performed. In an operation 1105, a DSO device (e.g., a health organization computing device 110 or a provider computing device 115) presents, on its display, the new patient with an electronic version of a first of one or more intake or consent forms. In an operation 1110, the DSO device prompts the patient to enter a first demographic identifier, which may for example be the patient's first name. Once the patient enters the first demographic identifier, in an operation 1115, the DSO device invokes a common key service to determine if there is a unique match for the patient in an MPI. If there is no match, in an operation 1120, the DSO device determines if there is an additional demographic identifier (e.g., middle initial, last name, zip code, date of birth, gender, last four digits of social security number, phone number, email address, etc.) that the patient may enter. If so, in operation 1125, the DSO device prompts the patient to enter a subsequent demographic identifier. The DSO device then may repeat the operations 1115-1125 until either a unique match is found in the MPI for the patient at operation 1115 or there is a determination that there is no additional demographic identifier for the patient to enter at operation 1120.

If a unique match is found at the operation 1115, at an operation 1130, the DSO device requests, from the common key service, the patient's common key and all additional information that the MPI has on the patient. If there is no additional demographic identifier to be entered, the DSO device invokes, at an operation 1135, an identity proofing service to determine if the identity of the patient may be verified. An identity proofing service is described in U.S. patent application Ser. Nos. 14/642,092 and 14/949,395, and may employ knowledge-based authentication and/or optional biometric identification. When either the patient's common key and MPI information are received at the operation 1130 or if the patient's identity is verified by the identity proofing service at the operation 1135, the DSO device prepopulates, at an operation 1140, as many unpopulated fields in the electronic form as possible using information from the MPI or the identity proofing service.

At an operation 1145, the DSO device invokes an ACRM system (e.g., ACRM system 105) that is used to provide an ACRS to determine, using either the patient's common key from the operation 1130 or the patient's verified identity from the operation 1135, if there is any known relationship between the patient and other providers where information about the patient may be stored. For every provider with which the patient has a relationship, the DSO device queries, at an operation 1150, the provider for information about the patient. The operation 1150 may involve retrieving an electronic address for the provider from a provider directory utility and invoking an intelligent query broker (as described in U.S. patent application Ser. No. 15/855,319, which is incorporated herein in its entirety) to query the provider at its electronic address for information about the patient.

Once additional information about the patient is received from other provider(s), the DSO device prepopulates as many remaining unpopulated fields in the electronic form as possible at an operation 1155. At an operation 1160, the DSO device prompts the patient to populate any remaining fields and/or correct any prepopulated fields. The operation 1160 may also be reached from the operation 1135 when the identity of the patient may not be verified by the identity proofing service.

At an operation 1165, the DSO device determines if there is any additional intake or consent form for the patient to populate. If there is any additional form, the DSO device loops back to operation 1155 and prepopulates as many fields as possible in the new form using information obtained from the MPI, the patient's identity verification, or provider(s) with the which patient has a relationship. Then, at the operation 1160, the DSO device prompts the patient to populate any remaining fields. If there is no additional form for the patient to populate at the operation 1165, the patient intake process 1100 is completed at an operation 1170. The patient intake process 1100 thus allows a patient to create intake forms once and enter every piece of information once, allowing secure communication and accurate sharing of the patient's information regardless of geographical location of health organizations or providers.

At this point it should be noted that intaking a patient at a provider in accordance with the present disclosure as described above may involve the processing of input data and the generation of output data to some extent. This input data processing and output data generation may be implemented in hardware or software. For example, specific electronic components may be employed in a computer server or similar or related circuitry for implementing the functions associated with intaking a patient at a provider in accordance with the present disclosure as described above. Alternatively, one or more processors operating in accordance with instructions may implement the functions associated with intaking a patient at a provider in accordance with the present disclosure as described above. If such is the case, it is within the scope of the present disclosure that such instructions may be stored on one or more non-transitory processor readable storage media (e.g., a magnetic disk or other storage medium), or transmitted to one or more processors via one or more signals embodied in one or more carrier waves.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Further, although the present disclosure has been described herein in the context of at least one particular implementation in at least one particular environment for at least one particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

1. A method for intaking a patient at a provider according to a set of instructions stored on a memory of a computing device and executed by a processor of the computing device, the method comprising the steps of:
presenting, on a display of the computing device, an electronic form including a plurality of fields to be populated with information about the patient;
prompting the patient to serially enter, via one of a graphical user interface and a keyboard, a plurality of demographic identifiers;
determining, after the patient enters each of the plurality of demographic identifiers and based on the one or more demographic identifiers entered, a match in a master person index between the patient and an identity of a person stored in the master person index;
requesting, from the master person index, a common key identifier pertaining to the patient and information about the patient; and
prepopulating one or more of the plurality of fields with information from the master person index.

2. The method of claim 1, wherein the requesting step includes invoking a common key service.

3. The method of claim 1, further comprising:
determining, based on the common key identifier, a relationship of the patient with one or more other providers;
querying the one or more other providers for additional information about the patient; and
prepopulating one or more of the plurality of fields with the additional information.

4. The method of claim 3, further comprising:
prompting the patient to populate empty fields out of the plurality of fields; and
prompting the patient to update the prepopulated fields.

5. The method of claim 3, wherein the determining step includes invoking an active care relationship service.

6. The method of claim 3, wherein the querying step includes:
retrieving, from a provider directory utility, one or more electronic addresses for the one or more other providers; and
invoking an intelligent query broker to query the one or more other providers for information about the patient.

7. The method of claim 1, further comprising:
prompting the patient to populate empty fields out of the plurality of fields; and
prompting the patient update the prepopulated fields.

8. The method of claim 1, further comprising invoking an identity proofing service to verify the identity of the patient.

9. The method of claim 1, wherein the electronic form is one of an intake form and a consent form, wherein
the intake form relates to one of the patient's medical history, prescriptions, HIPAA (Health Insurance Portability and Accountability Act) acknowledgment, release of information, financial policy, right of access, and health risk assessment; and
the consent form relates to one of mental/behavioral health, research, clinical trial, and advance directives.

10. The method of claim 1, wherein the computing device is one of a desktop computer, a laptop computer, a tablet, a smartphone, and a server.

11. A non-transitory computer readable medium having instructions stored thereon that, upon execution by a processor of a computing device, causes the computing device to perform operations, wherein the instructions comprise:
presenting, on a display of the computing device, an electronic form including a plurality of fields to be populated with information about a patient at a provider;
prompting the patient to serially enter, via one of a graphical user interface and a keyboard, a plurality of demographic identifiers;
determining, after the patient enters each of the plurality of demographic identifiers and based on the one or more demographic identifiers entered, a match in a master person index between the patient and an identity of a person stored in the master person index;
requesting, from the master person index, a common key identifier pertaining to the patient and information about the patient; and
prepopulating one or more of the plurality of fields with information from the master person index.

12. The non-transitory computer readable medium of claim 11, wherein the requesting instruction includes invoking a common key service.

13. The non-transitory computer readable medium of claim 11, wherein the instructions further comprise:
determining, based on the common key identifier, a relationship of the patient with one or more other providers;
querying the one or more other providers for additional information about the patient; and
prepopulating one or more of the plurality of fields with the additional information.

14. The non-transitory computer readable medium of claim 13, wherein the instructions further comprise:
prompting the patient to populate empty fields out of the plurality of fields; and
prompting the patient to update the prepopulated fields.

15. The non-transitory computer readable medium of claim 13, wherein the determining instruction includes invoking an active care relationship service.

16. The non-transitory computer readable medium of claim 13, wherein the querying instruction includes:
retrieving, from a provider directory utility, one or more electronic addresses for the one or more other providers; and
invoking an intelligent query broker to query the one or more other providers for information about the patient.

17. The non-transitory computer readable medium of claim 11, wherein the instructions further comprise:
prompting the patient to populate empty fields out of the plurality of fields; and
prompting the patient update the prepopulated fields.

18. The non-transitory computer readable medium of claim 11, wherein the instructions further comprise invoking an identity proofing service to verify the identity of the patient.

19. The non-transitory computer readable medium of claim 11, wherein the electronic form is one of an intake form and a consent form, wherein
the intake form relates to one of the patient's medical history, prescriptions, HIPAA (Health Insurance Portability and Accountability Act) acknowledgment, release of information, financial policy, right of access, and health risk assessment; and
the consent form relates to one of mental/behavioral health, research, clinical trial, and advance directives.

20. The non-transitory computer readable medium of claim 11, wherein the computing device is one of a desktop computer, a laptop computer, a tablet, a smartphone, and a server.
